(19) United States
(12) Patent Application Publication
(10) Pub. No.: US 2020/0261154 A1
(43) Pub. Date: Aug. 20, 2020

(54) PERIPHERAL VESSEL TISSUE MODIFICATION DEVICES AND METHODS OF USE THEREOF

(71) Applicant: ASAHI INTECC CO., LTD., Seto-shi (JP)

(72) Inventors: Wayne Ogata, San Ramon, CA (US); Steven Meyer, Oakland, CA (US)

(21) Appl. No.: 16/795,253

(22) Filed: Feb. 19, 2020

Related U.S. Application Data
(60) Provisional application No. 62/807,574, filed on Feb. 19, 2019.

(51) Int. Cl. A61B 18/24; A61B 18/12; A61B 18/14

(52) U.S. Cl. CPC A61B 18/245; A61B 18/1206; A61B 18/14; A61B 2018/0041; A61B 2018/142; A61B 2018/1417; A61B 2018/1475; A61B 2018/126

(57) ABSTRACT

A peripheral vessel tissue modification system includes a first longitudinal member, a second longitudinal member, and a radiofrequency energy source. The first member is configured for advancement into a peripheral vessel of a patient, is coupled to the energy source, and extends along a length between a proximal end and a distal end. The first member includes an inner lumen extending along its length and a distal electrode located on an outer surface proximate to the distal end and electrically coupled to the energy source. The second member is configured for insertion into the vessel through the lumen, is coupled to the energy source, and extends along a length between a proximal end and a distal end including a tip electrode. The second member is moveable with respect to the first member to create a bipolar arrangement between the distal electrode and the tip electrode for delivery of radiofrequency energy.

This application claims the benefit of U.S. Provisional Patent Application Ser. No. 62/807,574, filed Feb. 19, 2019, which is hereby incorporated by reference in its entirety.

### FIELD

The present technology relates to peripheral vessel tissue modification devices and methods of use thereof.

### BACKGROUND

Chronic total occlusion (CTO) is the complete blockage of a vessel and may have serious consequences if not treated in a timely fashion. The blockage could be due to atheromatous plaque or old thrombus. Treatment of chronic total occlusions is particularly difficult in peripheral arteries. Radiofrequency energy solutions have been employed in the treatment of CTOs. However, such solutions are limited in application when dealing with peripheral arteries.

None of the available strategies have provided satisfactory results for the most challenging of the CTOs in peripheral arteries. In case of hard calcified occlusions, the revascularization procedure can be tedious and time consuming. Therefore, there is a need for improved systems and methods of ablating or disrupting the occlusive material in peripheral arteries that are safe, efficacious and fast. It would be beneficial to have alternate techniques and devices that would recanalize a CTO in a peripheral vessel, such as an artery, without the shortcomings of the current techniques.

### SUMMARY

A peripheral vessel tissue modification system includes a radiofrequency energy source. A first longitudinal member is configured for advancement into a peripheral vessel in a patient, is coupled to the radiofrequency energy source, and extends along a first length between a first proximal end and a first distal end. The first longitudinal member includes an inner lumen extending along the first length and a distal electrode located on an outer surface of the first longitudinal member proximate to the first distal end and electrically coupled to the radiofrequency energy source. A second longitudinal member is configured for insertion into the peripheral vessel through the lumen of the first longitudinal member and is coupled to the radiofrequency energy source. The second longitudinal member extends along a second length between a second proximal end and a second distal end which includes a tip electrode. The second longitudinal member is movable with respect to the first longitudinal member to create a bipolar arrangement between the distal electrode and the tip electrode for the delivery of radiofrequency energy.

A method for modifying tissue in a peripheral vessel in a patient includes providing a first longitudinal member extending along a first length between a first proximal end and a first distal end having a distal electrode located proximate thereto on an outer surface of the first longitudinal member. A second longitudinal member is inserted through an inner lumen of the first longitudinal member, the second longitudinal member extending along a second length between a second proximal end and a second distal end comprising a tip electrode, such that the first distal end and the second distal end are substantially aligned. The first longitudinal member and the second longitudinal member are advanced to a location in the peripheral vessel. The first longitudinal member and the second longitudinal member are coupled to an energy source such that the distal electrode and the tip electrode are electrically coupled to the radiofrequency energy source. Radiofrequency energy is applied to the distal electrode and the tip electrode to modify tissue in the peripheral vessel.

A peripheral vessel tissue modification system includes a radiofrequency energy source. A longitudinal member is configured for advancement into a peripheral vessel and is coupled to the radiofrequency energy source. The longitudinal member extends along a length between a proximal end and a distal end. The longitudinal member includes an inner lumen extending along the length. At least two electrodes are electrically coupled to the energy source and extend along the length of the inner lumen. The at least two electrodes have a tip located on an outer surface of the distal end of the longitudinal member proximate to the first distal end. The at least two electrodes are configured in a bipolar arrangement at the distal end of the longitudinal member for the delivery of radiofrequency energy.

A method for modifying tissue in a peripheral vessel includes advancing a longitudinal member into the peripheral vessel. The longitudinal member extends along a length between a proximal end and a distal end. The longitudinal member comprises an inner lumen extending along the length and at least two electrodes extending along the length of the inner lumen. The at least two electrodes have a tip located on an outer surface of the distal end longitudinal member proximate to the distal end. The at least two electrodes are configured in a bipolar arrangement at the distal end of the longitudinal member for the delivery of radiofrequency energy. The longitudinal member is coupled to a radiofrequency energy source, such that the at least two electrodes are electrically coupled to the radiofrequency energy source. Radiofrequency energy is applied to the at least two electrodes to modify tissue in the peripheral vessel.

This technology provides a number of advantages including providing more efficient and effective devices and methods for treating peripheral vessels, such as arteries. By way of example, the exemplary devices of the present technology may be used for (1) vaporizing and removing soft or mature thrombus, with or without local catheter aspiration; (2) deep venous arterialization (DVA), facilitating the connection of a distal peripheral artery to an adjacent vein to shunt arterial blood to the venous network (e.g., within the foot); (3) re-entry from the subintimal lumen space into the distal true lumen; and/or (4) modifying intimal tissue and/or intimal and/or medial calcium within the vessel to enhance percutaneous transluminal angioplasty (PTA) balloon expansion and/or drug coated balloon (DCB)/drug eluting stent (DES) drug penetration/uptake via shockwave energy.

### BRIEF DESCRIPTION OF THE DRAWINGS

FIG. 1 is a partial top view and partial block diagram of an example of a peripheral vessel tissue modification system.

FIG. 2 is a cross-sectional view of a first longitudinal member that may be employed in the example of the peripheral vessel tissue modification system illustrated in FIG. 1.

FIGS. 3A and 3B are longitudinal views of the first longitudinal member that may be employed in the example of the peripheral vessel tissue modification system illustrated in FIG. 1.

FIGS. 4A-4D are perspective views of a distal tip of another example of the first longitudinal member having two bipolar electrodes that may alternatively be employed in the example of the peripheral vessel tissue modification system illustrated in FIG. 1.

FIG. 5 is a perspective view of yet another example of a first longitudinal member that may be employed in the example of the peripheral vessel tissue modification system illustrated in FIG. 1.

FIG. 6 is a perspective phantom view of luer/connector that may be used in the peripheral vessel tissue modification system illustrated in FIG. 1.

### DETAILED DESCRIPTION

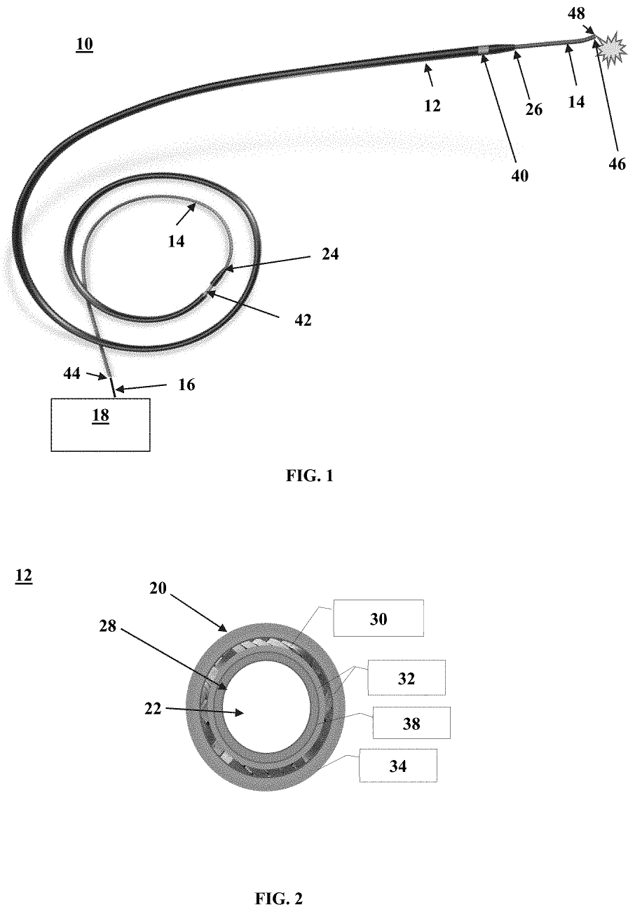

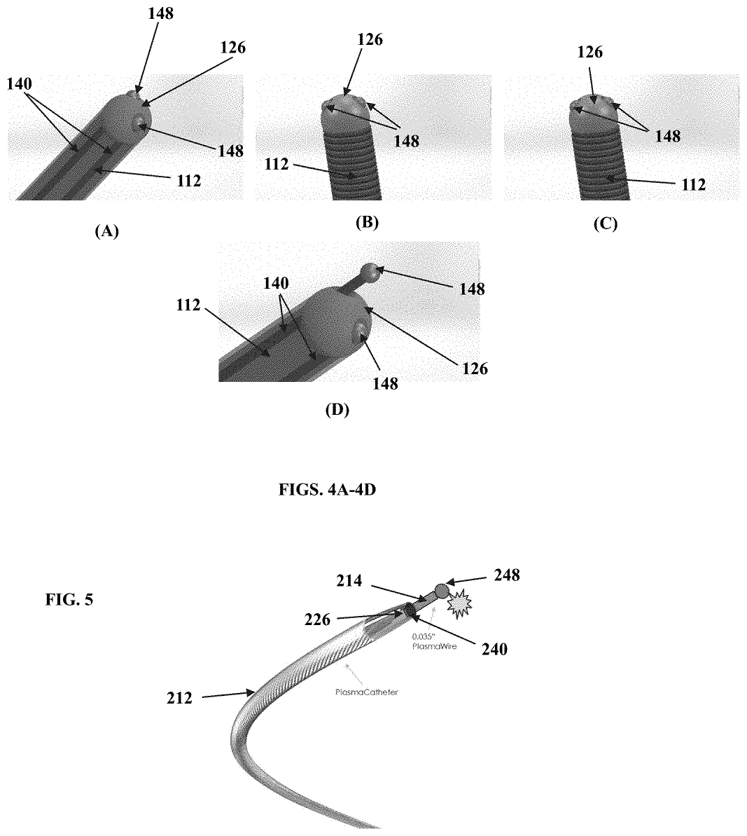

An example of a peripheral vessel tissue modification system 10 for modifying tissue and treating peripheral vessels such as arteries, veins, or other ducts in a patient is illustrated in FIG. 1. By way of example, the peripheral vessel tissue modification system 10 can be utilized for treating a femoral artery, popliteal artery, iliac artery, tibial artery, veins, or other ducts of the patient. In one example, the peripheral vessel tissue modification system 10 is utilized to treat below-the-knee (BTK) lesions, including chronic total occlusions (CTOs), using short bursts of bipolar radiofrequency (RF) energy. Although radiofrequency energy is described, other energy modalities, such as ultrasound, laser, or microwave energy, by way of example, may be utilized. The peripheral vessel tissue modification system 10 includes a first longitudinal member 12, a second longitudinal member 14, a coupler 16, and a radiofrequency energy source 18, although the tissue modification system 10 could include other types and/or numbers of elements, components, and/or devices in other configurations, such as illustrated by the additional examples described herein. By way of example, a first longitudinal member 112 as shown in FIG. 4 can be utilized such that the second longitudinal member 14 is not required. As another example, a first longitudinal member 212 can be used that is formed using a conventional catheter design. Other modifications to the system are also contemplated.

This exemplary technology provides a number of advantages including providing more efficient and effective tissue modification to treat peripheral arteries, veins, or ducts in a patient to facilitate intraluminal placement of conventional guidewires beyond peripheral chronic total occlusions. The devices of this technology are compatible with commercially available catheters and devices intended for use with, for example, 0.035″ diameter guidewires, and compatible with left or right femoral, radial, ulnar, or pedal patient vessel access techniques.

Referring more specifically to FIGS. 1-3, in this example the peripheral vessel tissue modification system 10 includes a first longitudinal member 12 that is configured to be advanced into the body of a patient, and into a peripheral vessel, such as femoral artery, popliteal artery, tibial artery, iliac artery, or a vein, by way of example, although the first longitudinal member 12 may be inserted into other peripheral vessels of the patient. In one example, the peripheral vessel may include an occlusion or a lesion that requires treatment.

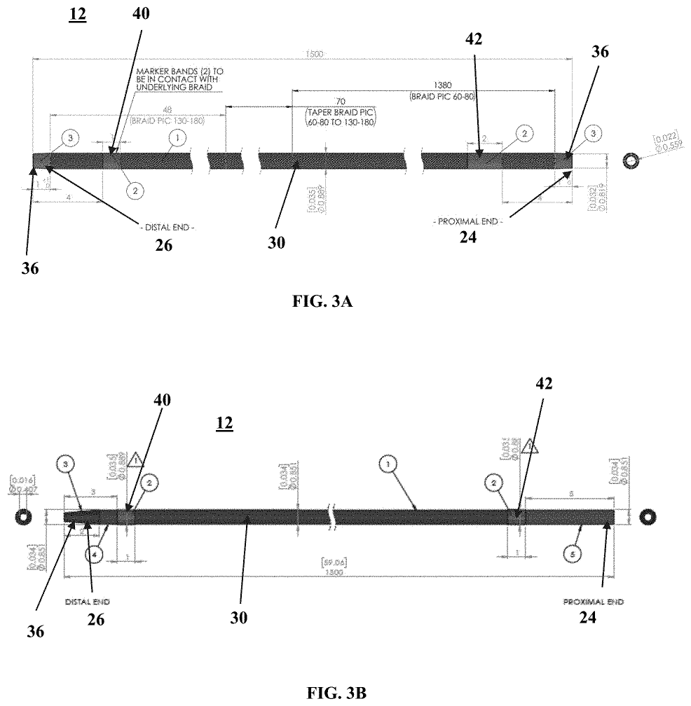

In this example, the first longitudinal member 12 is a hollow wire having an outer diameter 20 of about 0.035 inches or less, although the first longitudinal member 12 may have other dimensions. For purposes of this disclosure, the term “about” means±10%, ±5%, ±4%, ±3%, ±2%, or ±1%, when used to modify any stated values. The outer diameter 20 of the first longitudinal member 12 is configured for insertion into peripheral vessels of the patient, such as a peripheral artery. Referring more specifically to FIGS. 2 and 3, the first longitudinal member 12 includes a lumen 22 extending between a proximal end 24 and a distal end 26 of the first longitudinal member 12. In one example, the first longitudinal member 12 has a length extending between the proximal end 24 and the distal end 26 of about 90-150 cm, although the first longitudinal member 12 may have other lengths depending on the application.

Lumen 22 provides an inner diameter 28 of the first longitudinal member 12. The inner diameter 28 is sized to allow for insertion of the second longitudinal member 14, by way of example, although the lumen 22 may receive other types and/or numbers of other elements, such as other catheters, guidewires, microcatheters, or probes. In this example, the inner diameter 28 is configured to allow for passage of an element having a diameter of about 0.014 inches, although the inner diameter 28 may have other dimensions.

Referring now more specifically to FIG. 2, in this example the first longitudinal member 12 is constructed using a coiled and/or braided core 30, although in other examples the first longitudinal member 12 may be constructed of other materials, such as a laser-cut hypotube, by way of example only. The use of braided wire or hypotube for the core 30 advantageously provides stiffness, torque, kink resistance, and flexibility necessary to position the first longitudinal member 12 in the patient's peripheral arteries. The core 30 advantageously allows for sufficient push, steerability, and tracking of the first longitudinal member 12 to enable subsequent delivery of compatible catheters or other devices over the first longitudinal member 12 that may be used to treat the tissue.

In one example, the core 30 is a braided wire constructed from, for example, sixteen flat 304 stainless wires, with a per inch crossing (PIC) count of 40-80 near the proximal end 24 to give higher stiffness and greater push characteristics, transitioning to a higher PIC count of 130-180 on the distal end 26, to allow for greater flexibility, although the core 30 may be formed from other types and/or numbers of braided wires in other configurations, using other materials. In another example, the core 30 includes coiled wire having a varied pitch. Alternatively, in another example, the core 30 includes hypotube that is laser cut in a coiled or “notched” pattern, to achieve different flexibility characteristics along the length of the first longitudinal member 12.

In this example, the core 30 of the first longitudinal member 12 is surrounded by one or more dielectric layers 32. The dielectric layers 32 are constructed of a high strength dielectric material, such as polytetrafluoroethylene (PTFE) or polyimide, for example, although other high strength dielectric materials may be employed for the dielectric layers 32. The first longitudinal member 12 also includes an outer tubing 34 that provides the outer diameter 20 of the first longitudinal member 12. In this example, the outer tubing 34 is constructed of a thermoplastic, such as the polyether block amide PEBAX®, although other similar materials may be utilized for the outer tubing 34. In some examples, the outer tubing 34 does not cover the entire first longitudinal member 12. The outer tubing 34 allows for tips 36 to be molded over the ends of the first longitudinal member 12, as shown in FIG. 3A, to cover the ends of the core 30, which in some examples are braided wires. In one example, the tips 36 are tapered or radiused, as shown in FIG. 3B, for example. The tips 36 cover the ends of the braid, to provide atraumatic ends on the first longitudinal member 12.

Referring again to FIG. 2, the first longitudinal member 12 can optionally include an inner layer 38 that provides the inner diameter 28 of the lumen 22. The optional inner layer 38 is formed of a lubricous material, such PTFE, silicone, or a hydrophilic coating, by way of example only, to minimize friction and optimize movement of the second longitudinal member 14 with the lumen 22 of the first longitudinal member 12, as described in further detail below. In some examples, the outer tubing 34 is also coated with a lubricous material, such as silicone, PTFE, or a hydrophilic coating, to reduce friction when the first longitudinal member 12 is introduced to the patient's peripheral artery through a catheter.

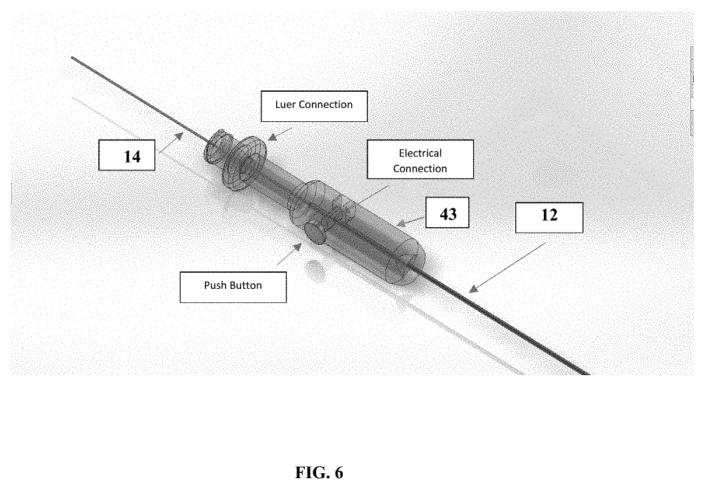

Referring now to FIG. 1, the first longitudinal member 12 includes an electrode 40 located proximate to the distal end 26. The electrode 40 is in electrical communication with the core 30, which is a braided wire or hypotube. The electrode 40 can serve as a radiopaque marker for identifying the distal end 26 of the first longitudinal member 12 under fluoroscopy, for example. The first longitudinal member 12 also includes a proximal contact 42 located at the proximal end 24. The proximal contact 42 provides an electrical connection to the core 30 and the electrode 40. The proximal end 24 of the first longitudinal member 12 can be coupled through coupler 16 to the radiofrequency energy source 18. By way of example, the proximal end 24 of the first longitudinal member 12 can be connected to the coupler 16 by a removable luer/connector 43, as shown in FIG. 6.

The second longitudinal member 14 is constructed as a standard guidewire as known in the art. In this example, the second longitudinal member 14 is configured for insertion into the lumen of the first longitudinal member 12. In one example, the second longitudinal member 14 has an outer diameter of about 0.014 inches for insertion into the lumen 22 of the first longitudinal member.

The second longitudinal member 14 includes a solid, taper ground stainless steel core wire. The second longitudinal member 14 extends along a length between a proximal end 44 and a distal end 46. The proximal end 44 of the second longitudinal member 14 is configured to be coupled to the radiofrequency energy source 18 through coupler 16, although other types of coupling systems may be used. In one example, the second longitudinal member 14 includes a flexible coil on the distal end 46 thereof. The distal end terminates at a tip 48 that may be configured as a concave or tapered portion of the second longitudinal member 14, or a ball tip that provides a reduced area that is configured to generate a higher current density at the tip 48 during operation.

The outer surface of the second longitudinal member 14 is coated or covered with a high dielectric strength material such as polyimide or PTFE that electrically insulates the core wire. The proximal end 44 and the distal end 46 of the second longitudinal member 14 are exposed areas of the core wire to allow for electrical connection to radiofrequency energy source 18 through the coupler 16 and to provide radiofrequency energy to the distal electrode.

Referring again to FIG. 1, in this example, both the first longitudinal member 12 and the second longitudinal member 14 are configured to be coupled to the radiofrequency energy source 18 by the coupler 16. In this example, the coupler 16 is a standard connector cable that includes inputs to couple to both the first and second longitudinal members 12 and 14. In one example, the first longitudinal member 12, which is hollow, is coupled to the one input of the coupler 16 using a removable luer/connector with an electrical contact that allows coupling between the coupler 16 and the first longitudinal member 12. The second longitudinal member 14 can be inserted directly into an input of the coupler 16. The coupler 16 electrically couples the output signals of the radiofrequency energy source 18 to the first longitudinal member 12 and the second longitudinal member 14.

In this example, radiofrequency energy source 18 is a radiofrequency generator that serves as a source of RF energy to be provided to the first longitudinal member 12 and the second longitudinal member 14 during operation. Optionally, in one example the radiofrequency energy source 18 is a hand-held battery-operated device, although other types of RF generators may be utilized. While the use of RF energy from radiofrequency energy source 18 for the purpose of ablation is described herein, it should be noted that other energy modalities may be used as well, for example ultrasound. In one example, one or both of first longitudinal member 12 and second longitudinal member 14 of the exemplary peripheral artery tissue modification system 10 of the present technology comprise one or more ultrasound transducers, instead of or in addition to RF electrodes as described below. The ultrasound transducers provide ultrasound energy for ablating an occlusion. Other energy modalities could include microwave and laser, although additional energy modalities known in the art may be employed.

An example of a method for peripheral vessel tissue modification utilizing radiofrequency energy will now be described with reference to FIG. 1. First, the first longitudinal member 12 is connected to the removable luer/connector 43 (as shown in FIG. 6) at the proximal end 24. The first longitudinal member 12 is then flushed with saline to remove air from the lumen 22.

Next, the second longitudinal member 14 is inserted into the lumen 22 of the first longitudinal member 14. The second longitudinal member 14 is inserted such that the distal tip 48 of the second longitudinal member 14 is substantially aligned with the distal end 26 of the first longitudinal member 12. In one example, the inner layer 38 of the first longitudinal member 12 is a lubricous layer that allows for easier insertion of the second longitudinal member 14 through the lumen 22.

The first longitudinal member 12 and the second longitudinal member 14 are then advanced together into the body of a patient, and into a peripheral vessel, such as the superficial femoral artery, the popliteal artery, the iliac artery, or the tibial artery, by way of example, although the first longitudinal member 12 and the second longitudinal member 14 can be advanced into other peripheral vessels, such as veins or ducts. The first longitudinal member 12 and the second longitudinal member 14 can be inserted into an optional support catheter or a percutaneous transluminal angioplasty (PTA) balloon catheter that is compatible with the outer diameter 20 of the first longitudinal member 12. By way of example the support catheter or PTA balloon catheter may be configured to receive the first longitudinal member 12 with the outer diameter 20 being about 0.035 inches. The support catheter is used to provide support and stability for the first longitudinal member 12 and the second longitudinal member 14 both prior to delivery, and during the delivery, of radiofrequency energy as described below.

The first longitudinal member 12 and the second longitudinal member 14 with the support catheter can be inserted to the body region, such as a peripheral vessel for treatment, through a sheath or guide catheter. In one example, the first longitudinal member 12 and the second longitudinal member 14 are advanced into a peripheral vessel, such as an artery, that includes an occlusion or a lesion that requires treatment. The position of the first longitudinal member 12 can be tracked using the distal electrode 40 as a radiopaque marker for identifying the distal end 26 of the first longitudinal member 12 under fluoroscopy and/or intravascular ultrasound, for example. In this example, the first longitudinal member 12, which includes the second longitudinal member 14 located therein, can be tracked and directed to the site of the occlusion or lesion in the peripheral vessel.

When the first longitudinal member 12 and the second longitudinal member 14 are located proximate to the treatment site (e.g., an occlusion or lesion in a peripheral vessel), the first longitudinal member 12 is extended from the optional support or PTA balloon catheter. In one example, the first longitudinal member 12 is extended at least about 5-10 mm out of the support catheter. In this configuration, the distal tip 48 of the second longitudinal member 14 remains substantially aligned with the distal end 26 of the first longitudinal member 12.

Next, with the first longitudinal member 12 extended out of the support catheter, the second longitudinal member 14 is advanced through the lumen 22 to extend out of the first longitudinal member 12. In one example, the distal tip 48 of the second longitudinal member 14 is extended between about 5-50 mm from the distal end 26 of the first longitudinal member 12. In another example, the second longitudinal member 14 can optionally be positioned inside the first longitudinal member 12, at or proximate to the distal end 26 of the first longitudinal member 12, such that during radiofrequency activation, as described below, the generated plasma and resultant shockwave energy are directed out of the distal end 26 of the first longitudinal member 12. In one example, the peripheral artery tissue modification system 10 is utilized with a PTA balloon catheter and is used to enhance the balloon inflation effects by delivering shockwave energy into the vessel wall of the peripheral artery, by way of example.

The position of the second longitudinal member 14 can be tracked using fluoroscopy and/or intravascular ultrasound. Once the desired position of the second longitudinal member 14 is confirmed, the removable luer/connector 43 (as shown in FIG. 6) is engaged with the proximal end 44 of the second longitudinal member 14. The removable luer/connector 43 provides the ability to flush the inner lumen 22, provides an electrical connection to the proximal contact 42 of the first longitudinal member 12, and also mechanically fixes the relative position of the first longitudinal member 12 and the second longitudinal member 14, for example, by compressing the proximal end 24 of the first longitudinal member 12 about a portion of the second longitudinal member 14 that extends from the proximal end 24 of the first longitudinal member 12.

Next, the first longitudinal member 12 is coupled directly to an input of the coupler 16 or through an electrical lead (not shown) on the removable luer/connector 43. The proximal end 44 of the second longitudinal member 14 is inserted in another input of the coupler 16. In this configuration, both the first longitudinal member 12 and the second longitudinal member 14 are electrically coupled to the radiofrequency energy source 18.

Radiofrequency energy is then applied from the radiofrequency energy source 18 to both the first longitudinal member 12 and the second longitudinal member 14. With the second longitudinal member 14 positioned proximate to, at, or extended from the distal end 26 of the first longitudinal member 12, a bipolar arrangement is provided between the distal electrode 40 of the first longitudinal member 12 and the distal tip 48 of the second longitudinal member 14. In one example, the distal tip 48 of the second longitudinal member 14 is configured with a taper or as a ball tip, such that the distal tip 48 provides an area of reduced surface area compared to the distal electrode 40 of the first longitudinal member 12. As a result, a greater current density is generated at the distal tip 48 during radiofrequency activation. The greater current density at the distal tip 48 allows for the majority of tissue modification effects, based on the delivered energy, to be at the distal tip 48, which provides for a more accurate deliver of energy to the treatment site, such as occlusion or lesion.

In one example, radiofrequency energy is delivered from the radiofrequency energy source 18 in multiple cycles. The first longitudinal member 12 and/or the second longitudinal member 14 can optionally be advanced as the tissue is modified. In one example, the first longitudinal member 12 and/or the second longitudinal member 14 can be advanced to access the true distal lumen of the peripheral vessel, such as a peripheral artery.

Once the true distal lumen of the peripheral artery, for example, is accessed, a compatible (e.g., operable with a catheter having a diameter of about 0.035 inches) PTA balloon catheter and or DCB/DES delivery catheter can be tracked over the first longitudinal member 12 to treat the occlusion or lesion. Alternatively, the first longitudinal member 12 can be removed, while the second longitudinal member 14 remains in place, in order to advance a compatible (e.g., operable with a catheter having a diameter of about 0.014 inches) over the second longitudinal member 14 to the treatment site.

In yet another example, the first longitudinal member 12 can be removed and the second longitudinal member 14 can be advanced further distally to address lesions in smaller, more tortuous vessels, such as for example in a BTK procedure. The second longitudinal member 14 is advanced in an antegrade direction to the lesion site. An additional wire, configured in the same manner as the second longitudinal member 14 can then be advanced into the target peripheral artery, or other vessel, using a retrograde approach (e.g. using pedal artery access). The two wires could be tracked together using the combined retrograde/antegrade approach to form a bipolar arrangement. Radiofrequency energy can then be applied between the wires to modify the tissue between the two distal tips to reestablish true lumen blood flow through the lesion site, by way of example only.

FIG. 4A-D illustrates a distal end 126 of another exemplary first longitudinal member 112 that may be employed in the peripheral vessel tissue modification system 10. In this example, the second longitudinal member 14 is not required. The structure and operation of the peripheral vessel tissue modification system 10 is the same as described with reference to FIG. 1 when using first longitudinal member 112, except as discussed below.

In this example, the first longitudinal member 112 includes two bipolar electrodes 140 located on the distal end 126 thereof. In this example, the first longitudinal member 112 is a wire having an outer diameter of about 0.035 inches or less, although the first longitudinal member 112 may have other dimensions. The outer diameter of the first longitudinal member 112 is configured for insertion into peripheral vessels, such as arteries or veins, of the patient. In this example, the first longitudinal member 112 is formed from a laser cut hypotube or wire reinforced (e.g., braided and/or coiled) tubing, through which the two electrodes would be housed. In another example, the first longitudinal member 112 is formed as a flexible coil of wire, with the electrodes 140 formed as separate dielectrically insulated wires in the coil. In this example, the first longitudinal member 112 could utilize a more standard solid taper ground core wire to achieve the various mechanical performance properties, with the coil providing both flexibility properties as well as acting as the electrodes 140, with the distal tips 148 exposed and fixed, and the proximal ends terminating in two electrical contacts. The first longitudinal member 112 is constructed to have flexibility and handling similar to a guidewire.

The first longitudinal member 112 does not have a fixed proximal luer or electrical leads, which allows for tracking compatible PTA balloon catheters or support catheters over the first longitudinal member 112. In one example, a support catheter may be employed to stabilize the first longitudinal member prior to and during radiofrequency activation. In another example, the first longitudinal member is utilized with a PTA balloon catheter and the first longitudinal member 112 is utilized to enhance the balloon inflation effects by delivering shockwave energy into the vessel wall using the electrodes 140.

The electrodes 140 provide a bipolar arrangement and are separated by a dielectric barrier, such as polyimide, PTFE, PEEK, or ceramic, by way of example only, located at the distal end 126 of the first longitudinal member 112. The dielectric barrier allows delivered radiofrequency energy to be concentrated at the distal end 126 in order to propagate energy effects forward or laterally depending on the electrode configuration for tissue modification.

The electrodes 140 extend along the length of the first longitudinal member 112, as shown in FIGS. 4A and 4D, and are fully insulated within the body of the first longitudinal member 112. The diameter and stiffness of the electrodes 140 can be varied to alter the stiffness and/or flexibility of the first longitudinal member 112. The proximal ends of the electrodes 140 would be coupled to the radiofrequency energy source 18 through the coupler 16, as shown in FIG. 1, to allow for the delivery of energy to the treatment site.

The electrodes 140 can be fixed or moveable. In one example, the proximal ends of the electrodes 140 (if fixed) would terminate at two separate electrical contacts (one for each), so that the proximal end of the first longitudinal member 112 could be inserted into a connector on the coupler 16 to electrically couple to the radiofrequency energy source 18. Alternately, the electrodes 140 could protrude from the proximal end of the first longitudinal member 112, to allow for insertion of each into a single connector or two connectors on the coupler 16. The electrodes 140 could also be configured to allow the user to move one (as shown in FIG. 4D) or both of the electrodes 140 either a fixed or variable distance away from the distal end 126 of the first longitudinal member 112. The size and/or shape of the exposed distal tip 148 of the electrodes 140 could be the same, or intentionally different to concentrate the radiofrequency energy effects on one electrode.

FIG. 5 illustrates another exemplary first longitudinal member 212 that may be employed in the exemplary peripheral vessel tissue modification system 10 illustrated in FIG. 1. The structure and operation of the peripheral vessel tissue modification system 10 is the same as described with reference to FIG. 1 when using first longitudinal member 212, except as discussed below.

In this example, the first longitudinal member 212 is a catheter. The structure and operation of the first longitudinal member 212 would be similar to the first longitudinal member 12, as described with respect to FIG. 1, except the first longitudinal member 212 includes a catheter design, including a fixed proximal luer hub (not shown). In this example, the first longitudinal member 212 is constructed of coil and/or braided wire reinforced tubing, with the wire acting as the electrical conduit, and insulating dielectric materials that form the inner and outer diameters of the first longitudinal member 212. In this example, the first longitudinal member 212 could include other catheter elements, such as an integrated PTA or low pressure balloon, or other expandable elements located at the distal end 226 to allow for greater stability within the peripheral vessel during radiofrequency energy delivery.

The first longitudinal member 212 can be a ≤4F profile catheter, by way of example, although other catheter sizes could be employed. The second longitudinal member 214 in this example would be the same as the second longitudinal member 14, but scaled up to have an outer diameter of about 0.035 inches, by way of example, to be compatible with the ≤4F profile catheter.

The first longitudinal member 212 includes an electrode 240 located near the distal end 226 thereof. The distal end 226 may also optionally be constructed of a conductive material (e.g., stainless steel) to act as the distal electrode 240. In this example, the luer hub includes a lead wire or contact for electrical connection with the distal electrode 240. The luer hub also provides a luer port to allow saline flushing of the first longitudinal member 212.

In this example, the first longitudinal member 212 is advanced over the second longitudinal member 214 or a standard 0.035 inch standard guidewire. Then, once at the lesion site in the peripheral vessel, the second longitudinal member 214 would be positioned such that the distal tip 248 is approximately 5-50 mm distal to the distal end 226 of the first longitudinal member 212. The second longitudinal member 214 could optionally be positioned at or proximal to the distal tip 248 of the first longitudinal member 212 (positioned inside), such that during radiofrequency activation, the generated plasma and resultant shockwave energy are directed out of the distal end 226 of the first longitudinal member 212.

The first and second longitudinal members 212 and 214 are coupled to the radiofrequency energy source 18 through the coupler 16, which may be a connector cable. When radiofrequency energy is applied from the radiofrequency energy source 18, the energy is focused at and around the distal tip 248 of the second longitudinal member 214. Similar to the first longitudinal member 12, as described with respect to FIG. 1, with the first longitudinal member 212 exchanged for a standard support catheter and the second longitudinal member 214, delivered in an antegrade direction, left in place, a second wire could be advanced toward the lesion and the second longitudinal member 214 via a retrograde approach. Radiofrequency energy can then be applied between the tips of the two wires to provide treatment.

Accordingly, as illustrated and described by way of the examples herein this technology provides more efficient and effective devices and methods for efficient and effective tissue modification to treat peripheral arteries to facilitate intraluminal placement of conventional guidewires beyond peripheral artery chronic total occlusions

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Accordingly, the invention is limited only by the following claims and equivalents thereto.

What is claimed is:

1. A peripheral vessel tissue modification system comprising:
an energy source;
a first longitudinal member configured for advancement into a peripheral vessel of a patient, the first longitudinal member coupled to the energy source and extending along a first length between a first proximal end and a first distal end, wherein the first longitudinal member comprises:
an inner lumen extending along the first length; and
a distal electrode located on an outer surface of the first longitudinal member proximate to the first distal end and electrically coupled to the radiofrequency energy source; and
a second longitudinal member configured for insertion into the peripheral vessel through the lumen of the first longitudinal member, the second longitudinal member coupled to the energy source and extending along a second length between a second proximal end and a second distal end comprising a tip electrode, wherein the second longitudinal member is movable with respect to the first longitudinal member to create a bipolar arrangement between the distal electrode and the tip electrode for the delivery of radiofrequency energy.

2. The system of claim 1, wherein the first longitudinal member has an outer diameter of about 0.035 inches.

3. The system of claim 2, wherein the first longitudinal member has an inner diameter of about 0.014 inches.

4. The system of claim 1, wherein the first longitudinal member comprises a braided or coiled wire core, or a hypotube core.

5. The system of claim 1, wherein the second longitudinal member is configured to be extended beyond the first longitudinal member by about 5-50 mm.

6. The system of claim 1, wherein the second longitudinal member is configured to be positioned inside or at the distal end of the first longitudinal member.

7. The system of claim 1, wherein the second longitudinal member has an outer diameter of about 0.014 inches.

8. The system of claim 1, wherein the tip electrode has a concave, tapered, or ball-shaped configuration.

9. A method for modifying tissue in a peripheral vessel of a patient, the method comprising:
providing a first longitudinal member extending along a first length between a first proximal end and a first distal end having a distal electrode located proximate thereto on an outer surface of the first longitudinal member;
inserting a second longitudinal member through an inner lumen of the first longitudinal member, the second longitudinal member extending along a second length between a second proximal end and a second distal end comprising a tip electrode, such that first distal end and the second distal end are substantially aligned;
advancing the first longitudinal member and the second longitudinal member to a location in the peripheral vessel;
coupling the first longitudinal member and the second longitudinal member to an energy source such that the distal electrode and the tip electrode are electrically coupled to the radiofrequency energy source; and
applying radiofrequency energy to the distal electrode and the tip electrode to modify tissue in the peripheral vessel.

10. The method of claim 9, wherein the first longitudinal member has an outer diameter of about 0.035 inches.

11. The method of claim 10, wherein the first longitudinal member has an inner diameter of about 0.014 inches.

12. The method of claim 9, wherein the first longitudinal member comprises a braided or coiled wire core, or a hypotube core.

13. The method of claim 9 further comprising:
extending the second distal end beyond the first distal end to create a bipolar arrangement between the distal electrode and the tip electrode for the delivery of radiofrequency energy.

14. The method of claim 13, wherein the extending the second distal end beyond the first distal end to create a bipolar arrangement between the distal electrode and the tip electrode for the delivery of radiofrequency energy comprises extending the second distal end beyond the first distal end by about 5-50 mm.

15. The method of claim 9, further comprising:
retracting the second distal end into the first distal end to create a bipolar arrangement between the distal electrode and the tip electrode for the delivery of radiofrequency energy.

16. The method of claim 9, wherein the second longitudinal member has an outer diameter of about 0.014 inches.

17. The method of claim 9, wherein the tip electrode has a tapered, concave, or ball-shaped configuration.

18. The method of claim 9, wherein the applying is repeated a plurality of times.

19. The method of claim 9, wherein the peripheral vessel comprises a femoral artery, a tibial artery, an iliac artery, a popliteal artery, or a peripheral vein.

20. The method of claim 9, wherein the tissue comprises an occlusion or a lesion.

21. A peripheral artery tissue modification system comprising:
a radiofrequency energy source;
a longitudinal member configured for advancement into a peripheral vessel of a patient, the longitudinal member coupled to the energy source and extending along a length between a proximal end and a distal end, wherein the longitudinal member comprises:
an inner lumen extending along the length; and
at least two electrodes electrically coupled to the energy source and extending along the length of the inner lumen, the at least two electrodes having a tip located on an outer surface of the distal end of the longitudinal member proximate to the distal end, wherein the at least two electrodes are configured in a bipolar arrangement at the distal end of the longitudinal member for the delivery of radiofrequency energy.

22. The system of claim 21, wherein the longitudinal member has an outer diameter of about 0.035 inches.

23. The system of claim 21, wherein the first longitudinal member comprises a braided or coiled wire core, or a hypotube core.

24. The system of claim 21, wherein at least one of the at least two electrodes is extendable from the first longitudinal member.

25. The system of claim 21, wherein at least one of the at least two electrodes is retractable into the first longitudinal member.

26. A method for modifying tissue in a peripheral vessel in a patient, the method comprising:
advancing a longitudinal member into the peripheral vessel, the longitudinal member extending along a length between a proximal end and a distal end, wherein the longitudinal member comprises an inner lumen extending along the length and at least two electrodes extending along the length of the inner lumen, the at least two electrodes having a tip located on an outer surface of the distal end longitudinal member proximate to the distal end, wherein the at least two electrodes are configured in a bipolar arrangement at the distal end of the longitudinal member for the delivery of radiofrequency energy;
coupling the longitudinal member to a radiofrequency energy source, such that the at least two electrodes are electrically coupled to the radiofrequency energy source; and
applying radiofrequency energy to the at least two electrodes to modify tissue in the peripheral artery.

27. The method of claim 26, wherein the longitudinal member has an outer diameter of about 0.035 inches.

28. The method of claim 26, wherein the first longitudinal member comprises a braided or coiled wire core, or a hypotube core.

29. The method of claim 26, wherein the first longitudinal member comprises a hypotube core.

30. The method of claim 26, wherein at least one of the at least two electrodes is extendable from the first longitudinal member.

31. The method of claim 26, wherein at least one of the at least two electrodes is retractable into the first longitudinal member.

32. The method of claim 26, wherein the applying is repeated a plurality of times.

33. The method of claim 26, wherein the peripheral vessel comprises a femoral artery, a tibial artery, an iliac artery, a popliteal artery, or a peripheral vein.

34. The method of claim 26, wherein the tissue comprises an occlusion or a lesion.
